(19) United States
(12) Patent Application Publication
(10) Pub. No.: US 2020/0378938 A1
(43) Pub. Date: Dec. 3, 2020

(54) INDOOR RADON PREDICTION SYSTEM AND METHOD FOR RADON REDUCTION

(71) Applicant: BETTERLIFE CO., LTD., Suwon-si, Gyeonggi-Do (KR)

(72) Inventor: Jae Sung LEE, Seongnam-si, Gyeonggi-Do (KR)

(73) Assignee: BETTERLIFE CO., LTD., Suwon-si, Gyeonggi-Do (KR)

(21) Appl. No.: 16/463,362

(22) PCT Filed: Mar. 11, 2019
(86) PCT No.: PCT/KR2019/002775

§ 371 Date: May 22, 2019

(30) Foreign Application Priority Data
Apr. 30, 2018 (KR) 10-2018-0049682

(51) Int. Cl. G01N 33/00; G01W 1/02; G01T 1/185; G01T 1/16; G06Q 30/00; G06Q 50/26; G06N 5/02; G05B 19/401

(52) U.S. Cl. CPC G01N 33/0055; G01W 1/02; G01T 1/185; G01T 1/1603; G05B 2219/37008; G06Q 50/26; G06N 5/02; G05B 19/401; G05B 2219/40585; G06Q 30/018

(57) ABSTRACT

An indoor radon prediction system and method for radon reduction is disclosed, the system including: a soil environment measurement module installed in soil surrounding a specific indoor space, and measuring environmental information data of temperature and humidity for the soil surrounding the corresponding specific indoor space; an indoor environment measurement module installed in the specific indoor space and measuring environmental information data of temperature and humidity for the corresponding specific indoor space; an indoor radon measurement module installed in a specific indoor space and measuring radon concentration data of the corresponding specific indoor space; a Korea Meteorological Administration (KMA) weather station management server constructing a database (DB) of big data information for surrounding weather conditions of the specific indoor space, thereby storing and managing the DB; and an indoor radon prediction management server.

### TECHNICAL FIELD

The present invention relates to indoor radon prediction system and method for radon reduction.

### BACKGROUND ART

In general, radon (Rn) is a kind of radioactive gas that causes alpha decay with a half-life of 3.8 days. It has colorless, odorless, and inert properties. It flows into a room mainly through cracks of a building from a ground of the building. However, Rn is also generated from products that are produced in the uranium decay series and contained in cement, soil, and other interior and exterior materials used in the construction of a building, whereby Rn is also introduced into a room.

When this radon enters the lungs through breathing, it becomes a main cause of a cancer by killing cells in the lungs. Therefore, the World Health Organization (WHO) and the US Environmental Protection Agency (USEPA) have defined radon as a second major causative agent following smoking causing lung cancer and recommend that concentration of radon in indoor air be controlled. Radon is also present in outdoor air or groundwater, but about 95% of exposure to radon is via indoor air.

In other words, as radon is the heaviest gas on the earth, once it enters the room, it accumulates without escaping. Radon enters into the lungs through breathing and collapses therein, thereby releasing alpha radiation. The alpha radiation is a nucleus of helium (²He⁺) and is less permeable than beta or gamma rays, but its mass is relatively large, causing a destruction of the lung cells.

Meanwhile, in order to reduce the radon gas introduced into a room, it is necessary for residents to ventilate the room periodically. However, in the case of cold conditions in wintertime or nighttime, the ventilation is not likely to be performed properly, so the residents are exposed to the radon gas, thereby being seriously endangered.

In particular, in the case of classrooms where students are present in groups, systematic management of radon gas is not being carried out, whereby there is concern about health of the students. In order to solve these problems, in Korea, a relevant government authority has mandated through an amendment of School Health Law so that a facility that measures and reduces the radon gas should be run, thereby keeping the radon gas in each classroom of a first floor or lower to be 148 Bq/m³ or lower.

As a conventional technology for managing the radon gas in a room, there is an “indoor radon removal device equipped with air purifier,” which is disclosed in the Korean Patent No. 10-1569270 B1. In this technology, an air supply pipe capable of supplying fresh air to an indoor ceiling portion of a room or the like is installed, and an air discharge pipe for sucking and discharging the polluted air is installed on the floor. Here, the air supplied through the air supply pipe passes through the air purifier, and fresh air having passed through the air purifier is supplied into the room.

In addition, the ‘System for integrally managing radon reduction facilities’ disclosed in the Korean Patent No. 10-1650436 is a system that stratifies a plurality of radon reduction facilities into major classes, medium classes, and minor classes, thereby constructing database, wherein a plurality of radon reduction facilities is classified into a plurality of categories as the major classes, each category of the major classes is classified into a plurality of places as the medium classes, and each place of the medium classes is classified into a plurality of influence factors on radon as the minor classes. Then, plurality of radon reduction facilities is integrally managed sequentially by each unit of each category, each place, and each radon influence factor in a manner of sequentially selecting one category at a major class level, at least one place at a medium class level, and at least one influence factor on radon at a minor class level. Accordingly, the system systematically manages and controls the numerous radon reduction facilities scattered everywhere considering the characteristics of each site for danger of radon exposure.

The ‘automatic ventilation equipment for reducing radon concentration and the building having the same’ disclosed in Korean Patent Application Publication No. 10-2016-0024076 A includes a radon concentration measurement unit for measuring a radon concentration value present in an indoor space, and a ventilation unit for ventilating the indoor space so that the measured radon concentration value achieves a predetermined reference concentration value or a reference concentration increase rate.

In general, the radon gas reduction system, after a radon gas sensor is installed to detect (or measure, hereinafter collectively referred to as detect) the radon gas in each room or classroom, controls the ventilation equipment of each room or classroom depending on the concentration of the radon gas detected in each room or classroom, so that the concentration of radon gas in each room or classroom is kept to be the prescribed value or lower.

However, because the radon gas sensor for detecting radon gas is generally expensive, there is a problem that the total cost of the radon gas reduction system is rapidly increased when the radon gas sensor is installed in each room or classroom.

### DISCLOSURE

### Technical Problem

Accordingly, the present invention has been made keeping in mind the above problems occurring in the conventional art, and an object of the present invention is to provide indoor radon prediction system and method for radon reduction that predict current indoor radon concentration on the basis of information of big data and the like on weather situation of Korea Meteorological Administration (KMA). Here, the information also includes environmental data of temperature, humidity, and the like of the indoor and ground together with the values of the indoor radon measured in the past. Accordingly, indoor radon levels in schools and houses can be reduced effectively at a low cost.

### Technical Solution

In order to achieve the above-mentioned object, a first aspect of the present invention is to provide an indoor radon prediction system for radon reduction, the system including: a soil environment measurement module installed in soil surrounding a specific indoor space, and measuring environmental information data of temperature and humidity for the soil surrounding the corresponding specific indoor space; an indoor environment measurement module installed in the specific indoor space and measuring environmental information data of temperature and humidity for the corresponding specific indoor space; an indoor radon measurement module installed in the specific indoor space and measuring radon concentration data of the corresponding specific indoor space; a Korea Meteorological Administration (KMA) weather station management server constructing a database (DB) of big data information for surrounding weather conditions of the specific indoor space, thereby storing and managing the DB; and an indoor radon prediction management server receiving the radon concentration data for the corresponding specific indoor space measured for a certain period of time from the indoor radon measurement module, generating an annual standard graph of the radon average concentration by time for the corresponding specific indoor space on the basis of the received radon concentration data, reflecting the big data information about the surrounding weather conditions for the corresponding specific indoor space managed by the KMA weather station management server and the environmental information data measured from the soil environment measurement module and the indoor environment measurement module, respectively, in the produced annual standard graph of the radon average concentration by time, in addition, calculating the estimated radon measurement value applying a correction index for each preset environmental element, generating an hourly, daily, monthly, and yearly radon concentration prediction graph for the corresponding specific indoor space on the basis of the calculated estimated radon measurement value, and constructing a DB for the radon concentration prediction graph, thereby storing and managing the DB.

Here, the soil environment measurement module may include: a soil environment measurement module installed in the soil surrounding the specific indoor space and measuring environmental information data of temperature and humidity for the soil surrounding the corresponding specific indoor space; a wireless communication unit wirelessly transmitting environmental information data of temperature and humidity for the soil surrounding the corresponding specific indoor space measured from the soil environment measurement sensor unit; and a soil environment measurement controller receiving the environmental information data of temperature and humidity for the soil surrounding the corresponding specific indoor space measured from the soil environment measurement sensor unit in real time, thereby controlling operations of the wireless communication unit in order for the received environmental information data to be wirelessly transmitted to the indoor radon prediction management server.

Preferably, the indoor environment measurement module may include: an indoor environment measurement sensor unit installed in a specific indoor space and measuring environmental information data of temperature and humidity for the corresponding specific indoor space; a wireless communication unit wirelessly transmitting environmental information data of temperature and humidity for the specific indoor space measured from the indoor environment measurement sensor unit; and an indoor environment measurement controller receiving the environmental information data of temperature and humidity for the corresponding specific indoor space measured from the indoor environment measurement sensor unit in real time, thereby controlling operations of the wireless communication unit in order for the received environmental information data to be wirelessly transmitted to the indoor radon prediction management server.

Preferably, the indoor radon measurement module may include: an indoor radon measurement sensor unit installed in a specific indoor space and measuring radon concentration data of the corresponding specific indoor space; a wireless communication unit wirelessly transmitting the radon concentration data for the corresponding specific indoor space measured from the indoor radon measurement sensor unit; and an indoor radon measurement controller receiving the radon concentration data for the corresponding specific indoor space measured from the indoor radon measurement sensor unit in real time, thereby controlling operations of the wireless communication unit in order for the received radon concentration data to be wirelessly transmitted to the indoor radon prediction management server.

Preferably, the indoor radon measurement sensor unit may be composed of a pulsed ionization chamber radon measurement sensor.

Preferably, when the indoor radon measurement sensor unit measures radon, the indoor radon measurement controller may calculate the amount of fine dust for the corresponding specific indoor space through following equation 1 depending on presence or absence of a filter for separating radon progeny,

Total amount of radon=amount of pure radon+amount of radon progeny being attached to fine dust, (Equation 1)

wherein, the amount of pure radon is an amount of radon that is obtained by removing the amount of the radon progeny using a filter for separating the radon progeny when radon is measured, wherein the radon progeny is a substance produced when radon decays and is measured in a state of being attached to the fine dust.

Preferably, the big data information for the surrounding weather conditions of the specific indoor space stored and managed in the KMA weather management server may include at least one of information of temperature, humidity, atmospheric pressure, fine dust, rainfall, and snowfall.

Preferably, the indoor radon prediction management server may compare and analyze the calculated estimated radon measurement value and the actual radon measurement value measured from the indoor radon measurement sensor unit provided in the indoor radon measurement module with each other, and, when a difference between the two values is greater than the preset reference deviation value, may provide a management service to notify a regular calibration diagnosis time of the indoor radon measurement sensor unit provided in the indoor radon measurement module to the preset administrator terminal through the communication network.

Preferably, the indoor radon prediction management server may provide a management service so that the ventilation facility is able to operate corresponding to the radon deviation for the corresponding indoor space according to the produced hourly, daily, monthly, and yearly radon concentration prediction graph for the corresponding specific indoor space.

Preferably, the indoor radon prediction management server may calculate the estimated radon measurement value by following equation 2,

Estimated radon measurement value=standard radon concentration measurement value×correction environment index, (Equation 2)

wherein, the standard radon concentration measurement value is a standardized value on a 24-hour basis for the corrected radon concentration measurement value over 48 hours, the corrected radon concentration measurement value is calculated as “radon concentration measurement value×environment index”, and the radon concentration measurement value is calculated as “radon concentration measurement value×environment index”, the radon concentration measurement value is calculated as “number of alpha rays×radon concentration conversion index”, the environment index is calculated as “temperature deviation index+humidity deviation index+fine dust deviation index+atmospheric pressure deviation index+rainfall index+snowfall index”, the temperature deviation index is calculated as “(indoor temperature−outdoor temperature)×temperature deviation weight”, the humidity deviation index is calculated as “outdoor humidity×humidity weight+(indoor humidity−outdoor humidity)×humidity deviation weight”, the fine dust deviation index is calculated as “indoor fine dust×fine dust weight+(outdoor fine dust−indoor fine dust)×fine dust deviation weight (when indoor fine dust<outdoor fine dust)”, the atmospheric pressure deviation index is calculated as (outdoor atmospheric pressure−indoor atmospheric pressure)×atmospheric pressure deviation weight”, the rainfall index is calculated as “rainfall×radon influence index×rainfall weight”, and the snowfall index is calculated as “snowfall×radon influence index×snowfall weight”, and the correction environment index is calculated as (1−current environment index/standard environment index)×environment weight”.

Preferably, when the indoor fine dust>outdoor fine dust, the fine dust deviation index may be calculated as “indoor fine dust×fine dust weight”.

A second aspect of the present invention is to provide an indoor radon prediction method for radon reduction, as the method using a system including a soil environment measurement module, an indoor environment measurement module, an indoor radon measurement module, and an indoor radon prediction management server, the method including: step (a) of measuring environmental information data of temperature and humidity for soil surrounding specific indoor space through the soil environment measurement module; step (b) of measuring environmental information data of temperature and humidity for the specific indoor space through the indoor environment measurement module; step (c) of measuring radon concentration data of the specific indoor space through the indoor radon measurement module; step (d) of generating an annual standard graph of the radon average concentration by time for the corresponding specific indoor space on the basis of the radon concentration data of the corresponding specific indoor space measured for a certain period of time in step (c) through the indoor radon prediction management server; step (e) of reflecting the big data information about the surrounding weather conditions for the corresponding specific indoor space managed by an external Korea Meteorological Administration (KMA) weather station management server and the environmental information data measured in step (a) and step (b), respectively, into the annual standard graph of the radon average concentration by time produced in step (d) through the indoor radon prediction management server, in addition, calculating estimated radon measurement value applying a correction index for each preset environmental element; and step (f) of, after generating the hourly, daily, monthly, and yearly radon concentration prediction graph for the corresponding specific indoor space on the basis of the estimated radon measurement value calculated in step (e) through the indoor radon prediction management server, constructing a DB for the radon concentration prediction graph, thereby storing and managing the DB.

Preferably, in step (e), the big data information for the surrounding weather conditions of the specific indoor space stored and managed in the external KMA weather management server may include at least one of information of temperature, humidity, atmospheric pressure, fine dust, rainfall, and snowfall.

Preferably, after step (e), when the estimated radon measurement value calculated in step (e) and the actual radon measurement value measured from the indoor radon measurement sensor unit provided in the indoor radon measurement module are compared and analyzed through the indoor radon prediction management server, and, a difference between the two values is greater than the preset reference deviation value, the method may further include a step of providing a management service to notify a regular calibration diagnosis time of the indoor radon measurement sensor unit provided in the indoor radon measurement module to the preset administrator terminal through the communication network.

Preferably, after step (f), the method may further include, a step of providing a management service so that the ventilation facility is able to operate corresponding to the radon deviation for the corresponding indoor space according to the hourly, daily, monthly, and yearly radon concentration prediction graph for the corresponding specific indoor space produced in step (f) through the indoor radon prediction management server.

Preferably, in step (e), the indoor radon prediction management server may calculate the estimated radon measurement value according to following equation 3,

Estimated radon measurement value=standard radon concentration measurement value×correction environment index, (Equation 3)

wherein, the standard radon concentration measurement value is a standardized value on a 24-hour basis for the corrected radon concentration measurement value over 48 hours, the corrected radon concentration measurement value is calculated as “radon concentration measurement value×environment index”, the radon concentration measurement value is calculated as “number of alpha rays×radon concentration conversion index”, the environment index is calculated as “temperature deviation index+humidity deviation index+fine dust deviation index+atmospheric pressure deviation index+rainfall index+snowfall index”, the temperature deviation index is calculated as “(indoor temperature−outdoor temperature)×temperature deviation weight”, the humidity deviation index is calculated as “outdoor humidity×humidity weight+(indoor humidity−outdoor humidity)×humidity deviation weight”, the fine dust deviation index is calculated as “indoor fine dust×fine dust weight+(outdoor fine dust−indoor fine dust)×fine dust deviation weight (when indoor fine dust<outdoor fine dust)”, the atmospheric pressure deviation index is calculated as (outdoor atmospheric pressure−indoor atmospheric pressure)×atmospheric pressure deviation weight”, the rainfall index is calculated as “rainfall×radon influence index×rainfall weight”, and the snowfall index is calculated as “snowfall×radon influence index×snowfall weight”, and the correction environment index is calculated as (1−current environment index/standard environment index)×environment weight”.

Preferably, when the indoor fine dust>the outdoor fine dust, the fine dust deviation index may be calculated as “indoor fine dust×fine dust weight”.

### Advantageous Effects

According to the indoor radon prediction system and method for radon reduction of the present invention as described above, a current indoor radon concentration is predicted on the basis of information of big data and the like on weather situation of the KMA. Here, the information also includes environmental data of temperature, humidity, and the like of the indoor and ground together with the values of the indoor radon measured in the past. Consequentially, there is an advantage that indoor radon of the schools and houses can be reduced effectively at a low cost.

### DESCRIPTION OF DRAWINGS

FIG. 1 is an overall block diagram for illustrating an indoor radon prediction system for radon reduction according to an embodiment of the present invention.

FIG. 2 is a block diagram for illustrating in detail a soil environment measurement module applied to an embodiment of the present invention.

FIG. 3 is a block diagram for illustrating in detail an indoor environment measurement module applied to an embodiment of the present invention.

FIG. 4 is a block diagram for illustrating in detail an indoor radon measurement module applied to an embodiment of the present invention.

FIG. 5 is a block diagram for illustrating in detail an administrator terminal applied to an embodiment of the present invention.

FIG. 6 is an overall flowchart for illustrating an indoor radon prediction system for radon reduction according to an embodiment of the present invention.

FIGS. 7 to 12 are views in graphs each illustrating environmental indices influencing indoor radon prediction for radon reduction according to an embodiment of the present invention.

### BEST MODE

The above and other objects, features, and advantages of the present invention will become more apparent by describing in detail exemplary embodiments thereof with reference to the attached drawings, whereby those skilled in the art may easily implement the technical idea of the present invention. In the following description, well-known functions or constructions that may unnecessarily obfuscate the present invention will be omitted.

Terms including ordinals, such as first, second, etc., may be used to describe various elements, but the elements are not limited to these terms. The terms are used only for the purpose of distinguishing one component from another. For example, without departing from the scope of the present invention, the first component may be referred to as the second component, and similarly, the second component may also be referred to as the first component. The terms used in the present application are used only to describe a specific embodiment and are not intended to limit the present invention. The singular expressions include plural expressions unless the context clearly dictates otherwise.

The term used in the present invention are selected among general terms that are widely used at present while the functions of the present invention are considered, this may vary depending on the intention of an engineer working in the related art, the precedent, or the emergence of new technology, and the like. In addition, in certain cases, there may be a term selected arbitrarily by the applicant, in which case the meaning thereof will be described in detail in the description of the corresponding part of present invention. Therefore, the term used in the present invention should be defined on the basis of the meaning of the term and the entire contents of the present invention, not on the basis of the simple name of the term.

When an element is referred to as “including” an element throughout the specification, it is to be understood that the element may include other elements as well, without departing from the spirit or scope of the present invention unless specifically stated otherwise. In addition, the terms “part”, “module”, and the like in the specification mean units for processing at least one function or operation, which may be implemented in hardware or software or in a combination of hardware and software.

Hereinafter, embodiments of the present invention will be described in detail with reference to the accompanying drawings. However, the following embodiments of the present invention may be modified into various other forms, and the scope of the present invention is not limited to the following embodiments. The embodiments of the present invention are provided to enable those skilled in the art to more fully understand the present invention.

Each block of the accompanying block diagram and combinations of each step of the accompanying flowchart may also be performed by computer program instructions (execution engines), which may be stored in a general-purpose computer, special purpose computer, or a processor of other programmable data processing equipment. In addition, the instructions that are executed through the computer or the processor of the other programmable data processing equipment generate means for performing the functions described in each block of the block diagram or in each step of the flowchart. These computer program instructions may also be stored in a computer usable or computer readable memory capable of supporting a computer or the processor of other programmable data processing equipment to implement the function in a particular manner. Therefore the instructions stored in the computer usable or computer readable memory are possible to produce a manufacturing item containing instruction means for performing the function described in each block of the block diagram or in each step of the flowchart.

In addition, because the computer program instructions may also be loaded onto the computer or the other programmable data processing equipment, a series of operating steps is performed on a computer or other programmable data processing equipment, thereby creating a process that is executed by the computer. Here, the instructions executing the computer or the other programmable data processing equipment are also possible to provide steps to perform the functions described in each block of the block diagram and at each step of the flowchart.

In addition, each block or each step may represent a portion of a module, a segment, or a code that includes one or more executable instructions for executing the specified logical functions, and in some alternative embodiments, it should be noted that functions described in the blocks or the steps may occur to be out of order. For example, two successive blocks or steps may actually be performed substantially concurrently, and it is also possible that the blocks or steps are performed in the reverse order of the function as needed.

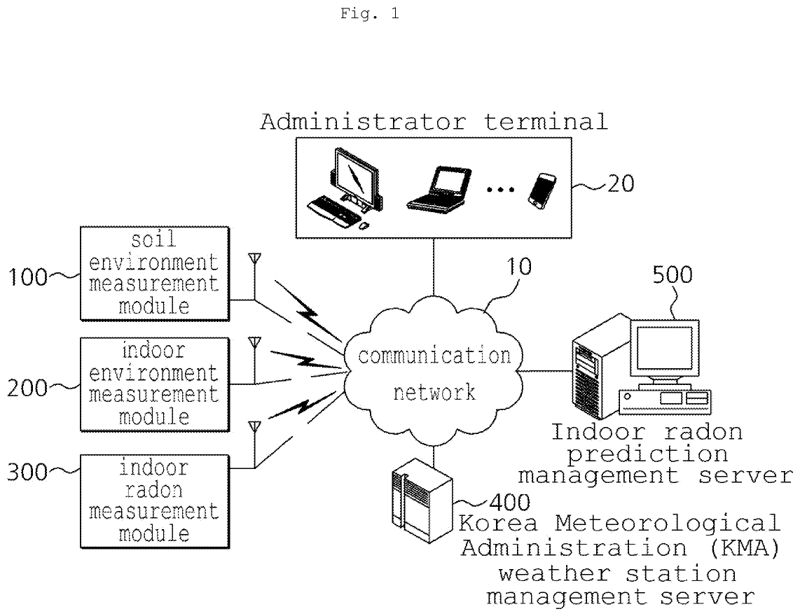

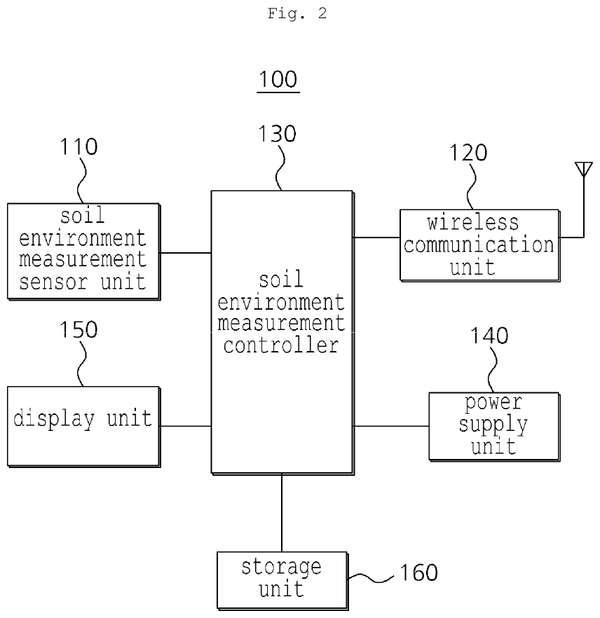

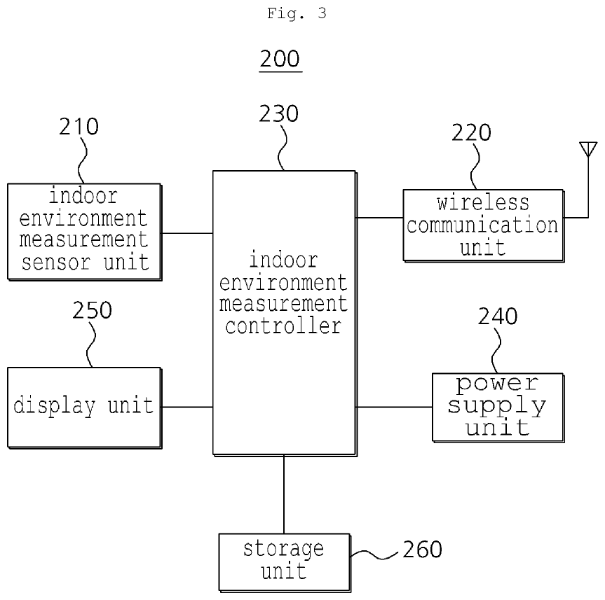

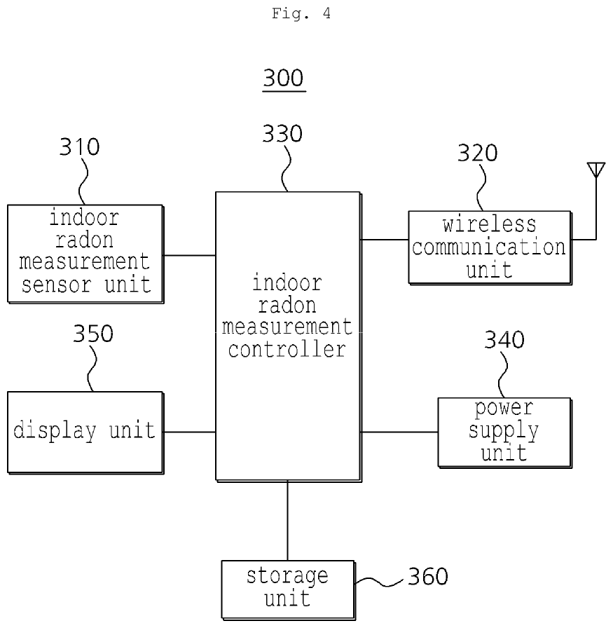

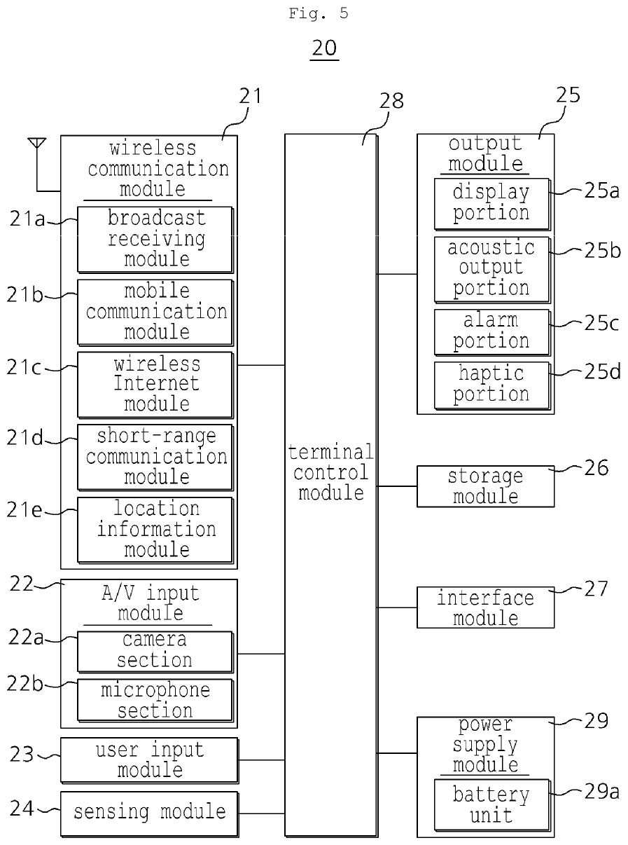

FIG. 1 is an overall block diagram for illustrating an indoor radon prediction system for radon reduction according to an embodiment of the present invention, FIG. 2 is a block diagram for illustrating in detail a soil environment measurement module applied to an embodiment of the present invention, FIG. 3 is a block diagram for illustrating in detail an indoor environment measurement module applied to an embodiment of the present invention, FIG. 4 is a block diagram for illustrating in detail an indoor radon measurement module applied to an embodiment of the present invention, and FIG. 5 is a block diagram for illustrating in detail a terminal control module applied to an embodiment of the present invention.

With reference with FIGS. 1 to 5, the indoor radon prediction system for radon reduction according to an embodiment of the present invention includes a soil environment measurement module 100, an indoor environment measurement module 200, an indoor radon measurement module 300, a Korea Meteorological Administration (KMA) weather station management server 400, and an indoor radon prediction management server 500, and the like. In addition, the indoor radon prediction system for radon reduction according to an embodiment of the present invention may further include an administrator terminal 20. Meanwhile, because elements illustrated in FIG. 1 are not essential, the indoor radon prediction system for radon reduction according to an embodiment of the present invention may have more or fewer elements than the elements illustrated in FIG. 1.

Hereinafter, the elements of the indoor radon prediction system for radon reduction according to an embodiment of the present invention will be described in detail.

The soil environment measurement module 100 is installed in the soil surrounding a specific indoor space (for example, a house, a building, a school, or the like), and performs a function of measuring environmental information data of temperature, humidity, and the like for the soil surrounding the corresponding specific indoor space.

As illustrated in FIG. 2, the soil environment measurement module 100 includes: a soil environment measurement sensor unit 110, a wireless communication unit 120, a soil environment measurement controller 130, a power supply unit 140, and the like. In addition, the soil environment measurement module 100 applied to an embodiment of the present invention may further include a display unit 150, a storage unit 160, and the like. Meanwhile, because elements illustrated in FIG. 2 are not essential, the soil environment measurement module 100 applied to an embodiment of the present invention may have more or fewer elements than the soil environment measurement module 100 illustrated in FIG. 2.

Hereinafter, the elements of the soil environment measurement module 100 applied to an embodiment of the present invention will be described in detail.

The soil environment measurement sensor unit 110 is installed in the soil surrounding the specific indoor space and performs a function of measuring environmental information data of temperature, humidity, and the like of the soil surrounding the corresponding specific indoor space.

The soil environment measurement sensor unit 110 preferably measures environmental information data of temperature, humidity, and the like of soil surrounding the corresponding specific indoor space but is not limited thereto. For example, environmental information such as the atmospheric pressure, the solar irradiance, the wind speed, the wind direction, soil moisture, soil salinity, or atmospheric fine dust may be measured.

That is, the soil environment measurement sensor unit 110 may include, for example, a temperature measurement sensor, a humidity measurement sensor, an atmospheric pressure measurement sensor, a solar irradiance measurement sensor, a wind speed measurement sensor, a wind direction measurement sensor, a soil moisture measurement sensor, a soil salinity measurement sensor, a fine dust sensors, and the like, thereby collecting and processing environmental information on the soil surrounding the specific indoor space.

At this time, the temperature measurement sensor is a sensor for measuring a temperature of an object to be measured such as air, water, or soil, and a thermistor element, whose internal resistance value changes according to the ambient temperature change, may be used. Here, the thermistor element may be a negative temperature coefficient (NTC) thermistor, a positive temperature coefficient (PTC) thermistor, or a critical temperature resistor (CTR) thermistor.

Such a temperature measurement sensor is preferably composed of contact-type temperature sensor using a thermistor element but is not limited thereto. For example, a thermocouple sensor, a bimetal, an IC temperature sensor, or an infrared sensor that is a noncontact-type temperature sensor may be used.

The humidity measurement sensor is a sensor for measuring the humidity of an object to be measured such as air or soil and normally measures the humidity using a change in the electrical property of the humidity-sensing substance by moisture.

Such humidity measurement sensors may be classified largely into resistance type humidity sensor and capacitance type humidity sensor and are widely applied to provide optimum conditions for automobile and medical devices, air purification systems, and automatic air-conditioning systems, as well as household appliances and mobile phones.

The resistance type humidity sensor measures the humidity using a change in resistance, which is changed, by humidity. This resistance type humidity sensor is widely used because it is relatively cost competitive as compared with the capacitance type humidity sensor.

However, in recent years, because the capacitance type humidity sensor is able to be manufactured in the form of a one chip on a semiconductor substrate, the capacitance type humidity sensor is able to secure a price competitiveness advantage over the resistance humidity sensor, and use thereof is being increased. Particularly, the capacitance type humidity sensor is superior to the resistance type humidity sensor in reliability, and has the advantage that the sensor characteristic is linear and the influence of temperature is small.

Such a capacitance type humidity sensor is a sensor using the principle that the capacitance is changed according to the amount of water molecules adsorbed on the humid membrane and operates in the form of a capacitor that uses humidity sensitive material, such as polyimide, ceramic, and the like whose dielectric permittivity dielectric constant changes when moisture is absorbed, as a dielectric. That is, the principle is to detect the capacitance changes due to the dielectric permittivity changes as the moisture permeates through the humidity sensing layer when there is a humidity sensing layer that detects humidity.

The atmospheric pressure measurement sensor is a sensor for measuring the atmospheric pressure, is generally composed of an element such as a piezoelectric element of which resistance, current or voltage changes according to an atmospheric pressure, and outputs the atmospheric pressure measurement value in an appropriate voltage value.

The solar irradiance measurement sensor is a sensor for measuring the solar radiation quantity in the atmosphere, is capable of measuring the light amount by sensing the amount of electricity generated by the charge value varying according to the amount of light, and is the solar radiation sensor type measuring about 0 to 1800 W/m² with an accuracy of about 5%.

The wind speed and wind direction measurement sensors are sensors for measuring the wind speed and wind direction in the atmosphere, are preferable to use a method of measuring the wind speed and wind direction by measuring the cooling effect of the wind with the temperature change using the resistive temperature detector (RTD), but are not limited thereto. That is, it is possible to measure the wind speed as well as to determine the wind direction vectorially according to the degree to which the piezo sensors vibrate by wind using a plurality of piezo sensors.

The soil moisture sensor is a sensor for measuring soil moisture status information such as soil water content and the like and oscillates high frequency signals in standard condensers having soil as a medium and air as a medium, respectively, thereby calculating respective electrostatic capacitance value according to the difference in capacitance permittivity. Here, the soil moisture sensor is preferably composed of a capacitance permittivity type soil moisture measurement sensor measuring the moisture content in the soil through a predetermined formula by measuring frequency or cycle of the high frequency signal according to the calculated capacitance value but is not limited thereto. For example, the soil moisture sensor may be composed of a measurement sensor using an electric resistance of an electrode in a gypsum block, a measurement sensor using neutron scattering, and the like. The soil moisture status information measured by such a soil moisture sensor may be a pF numerical value, a percentage (%), or a pressure unit, and the like.

The salinity measurement sensor is a sensor for measuring salinity (salinity concentration) of an object to be measured and uses the principle that the electrical conductivity appears differently depending on the amount of salinity. That is, it is possible to convert the measured electrical conductivity into the salinity concentration using the principle that the current flowing through the sample is inversely proportional to the resistance and proportional to the electrical conductivity.

The wireless communication unit 120 performs a function of wirelessly transmitting environmental information data of temperature, humidity, and the like for the soil surrounding the corresponding specific indoor space measured from the soil environment measurement sensor unit 110.

The wireless communication unit 120 may be implemented using wireless Internet communication method such as, for example, wireless LAN (WLAN) (Wi-Fi), wireless broadband (Wibro), World Interoperability for Microwave Access (Wimax), high speed downlink packet access (HSDPA), and wireless personal area network (WPAN) or may be implemented using a short-range wireless communication scheme such as, for example, Bluetooth, ultra wide band (UWB), radio frequency identification (RFID), infrared (IR) communication, and the like.

The soil environment measurement controller 130 controls the overall operation of the soil environment measurement module 100, may perform various functions for the soil environment measurement module 100, and may execute or perform a set of various software programs and/or instructions stored in the storage unit 160 for processing the data. That is, the soil environment measurement controller 130 may process various signals on the basis of the information stored in the storage unit 160.

In addition, the soil environment measurement controller 130 may transmit and receive various signals to and from the wireless communication unit 120. That is, the soil environment measurement controller 130 may perform various calculations on the basis of various signals transmitted and received to and from the wireless communication unit 120.

That is, the soil environment measurement controller 130 controls operations of the soil environment measurement sensor unit 110, the wireless communication unit 120, the display unit 150, the storage unit 160, and the like. In particular, the soil environment measurement controller 130 receives the environmental information data of temperature, humidity, and the like for the soil surrounding the corresponding specific indoor space measured from the soil environment measurement sensor unit 110 in real time, thereby performing a function of controlling operations of the wireless communication unit 120 in order for the received environmental information data to be wirelessly transmitted to the indoor radon prediction management server 500.

The power supply unit 140 performs a function of supplying power necessary for the respective units, that is, the soil environment measurement sensor unit 110, the wireless communication unit 120, the soil environment measurement controller 130, the display unit 150, the storage unit 160, and the like. Therefore, the power supply unit 140 is preferable to be implemented to convert power source (e.g., AC 220V) to a DC and/or AC power source for continuous power supply but is not limited thereto. For example, the power supply unit 140 may be implemented with a solar power supply or a conventional portable battery.

For a manager to be able to perform monitoring, the display unit 150 performs a function of displaying the environmental information data of temperature, humidity, and the like for the soil surrounding the corresponding specific indoor space, wherein the environmental information data is measured from the soil environment measurement sensor unit 110 according to the control of the soil environment measurement controller 130.

The display unit 150 may include at least one among a liquid crystal display (LCD), a light emitting diode (LED), a thin film transistor-liquid crystal display (TFT LCD), an organic light emitting diode (OLED), a flexible display, a plasma display panel (PDP), alternate lighting of surfaces (ALiS), digital light processing (DLP), a liquid crystal on silicon (LCoS), a surface-conduction electron-emission display (SED), a field emission display (FED), laser TV (quantum dot laser, liquid crystal laser), ferroelectric liquid display (FLD), interferometric modulator display (iMoD), thick film dielectric electricity (TDEL), quantum dot display (QD-LED), a telescopic pixel display (TPD), an organic light-emitting field-effect transistor (OLET), a laser phosphor display (LPD), and a 3D display, but is not limited thereto. For example, the display unit 150 may include any display capable of displaying a number, a character, or the like.

The storage unit 160 may include a program memory and a data memory. The program memory stores programs for controlling the general operation of the soil environment measurement module 100. At this time, the program memory may store a program for measuring environmental information data of temperature, humidity, and the like for the soil surrounding the corresponding specific indoor space through the soil environment measurement module 100.

In addition, the program memory may store a program for driving the soil environment measurement sensor unit 110, the wireless communication unit 120, the display unit 150, the storage unit 160, and the like under the control of the soil environment measurement controller 130. The data memory stores data generated during the execution of the programs in the soil environment measurement module 100. In the data memory, for example, device information, channel information, frequency information, network information, or the like may be stored. In addition, in the data memory of the storage unit 160, unique module identification information of the soil environment measurement module 100 may be stored.

In addition, the storage unit 160 may store the environmental information data of temperature, humidity, and the like for the soil surrounding the corresponding specific indoor space, wherein the environmental information data is measured from the soil environment measurement sensor unit 110 according to the control of the soil environment measurement controller 130.

That is, the storage unit 160 may store and maintain at least one program code executed through the soil environment measurement controller 130 and at least one type of data set that the program code uses.

The storage unit 160 may include at least one type of readable memory media among, for example, a flash memory type, a hard disk type, a multimedia card micro type, a card type memory (for example, an SD or XD memory), a random access memory (RAM), a static random access memory (SRAM), a read-only memory (ROM), an electrically erasable programmable read-only memory (EEPROM), a programmable read-only memory (PROM), a magnetic memory, a magnetic disk, an optical disk, and the like.

The indoor environment measurement module 200 is installed in a specific indoor space and performs a function of measuring environmental information data of temperature, humidity, and the like for the corresponding specific indoor space.

As illustrated in FIG. 3, the indoor environment measurement module 200 includes: an indoor environment measurement sensor unit 210, a wireless communication unit 220, an indoor environment measurement controller 230, a power supply unit 240, and the like. In addition, the indoor environment measurement module 200 applied to an embodiment of the present invention may further include a display unit 250, a storage unit 260, and the like. Meanwhile, the elements illustrated in FIG. 3 are not essential, the indoor environment measurement module 200 applied to an embodiment of the present invention may have more or fewer elements than the indoor environment measurement module 200 illustrated in FIG. 3.

Hereinafter, the elements of the indoor environment measurement module 200 applied to an embodiment of the present invention will be described in detail.

The indoor environment measurement sensor unit 210 is installed in a specific indoor space and performs a function of measuring environmental information data of temperature, humidity, and the like for the corresponding specific indoor space.

The indoor environment measurement sensor unit 210 preferably measures environmental information data of temperature, humidity, and the like for the specific indoor space, but is not limited thereto. For example, the indoor environment measurement sensor unit 210 may measure environmental information of atmospheric pressure, fine dust, and the like.

That is, the indoor environment measurement sensor unit 210 may include a temperature measurement sensor, a humidity measurement sensor, an atmospheric pressure measurement sensor, a fine dust measurement sensor, and the like, thereby collecting and processing environmental information for the specific indoor space.

The wireless communication unit 220 performs a function of wirelessly transmitting environmental information data of temperature, humidity, and the like for the specific indoor space measured from the indoor environment measurement sensor unit 210.

The indoor environment measurement controller 230 controls the overall operation of the indoor environment measurement module 200, may perform various functions for the indoor environment measurement module 200, and may execute or perform a set of various software programs and/or instructions stored in the storage unit 260 for processing the data. That is, the indoor environment measurement controller 230 may process various signals on the basis of the information stored in the storage unit 260.

In addition, the indoor environment measurement controller 230 may transmit and receive various signals to and from the wireless communication unit 220. That is, the indoor environment measurement controller 230 may perform various calculations on the basis of various signals transmitted and received to and from the wireless communication unit 220.

That is, the indoor environment measurement controller 230 controls the operation of the indoor environment measurement sensor unit 210, the wireless communication unit 220, the display unit 250, the storage unit 260, and the like. In particular, the indoor environment measurement controller 230 receives the environmental information data of temperature, humidity, and the like for the corresponding specific indoor space measured from the indoor environment measurement sensor unit 210 in real time, thereby performing a function of controlling operations of the wireless communication unit 220 in order for the received environmental information data to be wirelessly transmitted to the indoor radon prediction management server 500.

The power supply unit 240 performs a function of supplying power necessary for the respective units, that is, the indoor environment measurement sensor unit 210, the wireless communication unit 220, the indoor environment measurement controller 230, the display unit 250, the storage unit 260, and the like. Therefore, the power supply unit 240 is preferable to be implemented to convert power source (e.g., AC 220V) to a DC and/or AC power source for continuous power supply but is not limited thereto. For example, the power supply unit 240 may be implemented with a solar power supply or a conventional portable battery.

For a manager to be able to monitor, the display unit 250 performs a function of displaying the environmental information data of temperature, humidity, and the like for the corresponding specific indoor space, wherein the environmental information data is measured from the indoor environment measurement sensor unit 210 according to the control of the indoor environment measurement controller 230.

The storage unit 260 may include a program memory and a data memory. The program memory stores programs for controlling the general operation of the indoor environment measurement module 200. At this time, the program memory may store a program for measuring environmental information data of temperature, humidity, and the like for the corresponding specific indoor space through the indoor environment measurement module 200.

In addition, the program memory may store a program for driving the indoor environment measurement sensor unit 210, the wireless communication unit 220, the display unit 250, the storage unit 260, and the like under the control of the indoor environment measurement controller 230. The data memory stores data generated during the execution of the programs in the indoor environment measurement module 200. In the data memory, for example, module information, channel information, frequency information, network information, or the like may be stored. In addition, in the data memory of the storage unit 260, unique module identification information of the indoor environment measurement module 200 may be stored.

In addition, the storage unit 260 may store the environmental information data of temperature, humidity, and the like for the corresponding specific indoor space, wherein the environmental information data is measured from the indoor environment measurement sensor unit 210 according to the control of the indoor environment measurement controller 230.

That is, the storage unit 260 may store and maintain at least one program code executed through the indoor environment measurement controller 230 and at least one type of data set that the program code uses.

The indoor radon measurement module 300 is installed in a specific indoor space and performs a function of measuring radon concentration data of the corresponding specific indoor space.

As illustrated in FIG. 4, the indoor radon measurement module 300 includes: an indoor radon measurement sensor unit 310, a wireless communication unit 320, an indoor radon measurement controller 330, a power supply unit 340, and the like. In addition, the indoor radon measurement module 300 applied to an embodiment of the present invention may further include a display unit 350, a storage unit 360, and the like. Meanwhile, the elements illustrated in FIG. 4 are not essential, the indoor radon measurement module 300 applied to an embodiment of the present invention may have more or fewer elements than the indoor radon measurement module 300 illustrated in FIG. 4.

Hereinafter, the elements of the indoor radon measurement module 300 applied to an embodiment of the present invention will be described in detail.

The indoor radon measurement sensor unit 310 is installed in a specific indoor space and performs a function of measuring radon concentration data of the corresponding specific indoor space.

The indoor radon measurement sensor unit 310 is, for example, preferably composed of a pulsed ionization chamber radon measurement sensor, but is not limited thereto. For example, the indoor radon measurement sensor unit 310 may be a surface barrier detector, a high purity semiconductor (pure Ge) detector, a scintillation detector, a solid state junction counter, or the like as a device for detecting alpha particles.

That is, the pulsed ionization chamber radon measurement sensor is structured such that a probe-shaped electrode is installed at the inner center of a cylindrical box made of metal, and an electric field is formed by applying a bias voltage between the metal cylinder and the inner probe.

When alpha decay occurs and an alpha particles are emitted in the ionization chamber, alpha particles disappear by colliding with air, but ion charges are generated. Therefore, alpha particles may be detected by amplifying signals generated due to the ion charges absorbed through a central probe. The sensor itself consists of a metal cylinder and a probe, is very cheap, has good durability, and has nothing to do with light, thereby providing an advantage of enhancing air permeability.

As for the surface barrier detector, a depletion layer such as a p-n junction is formed on the surface of a semiconductor due to surface level, an oxide film, or the like, whereby the vicinity of the surface is an obstacle for charge transfer. Practically, gold is deposited on the surface of n-type Si at a thickness of about 100 μm/d, which is used as an electrode on one side and radiation is incident on an opposite surface. Here, the thickness of the depletion layer is about 50 to 500 μm, and because energy loss on the surface is small, it is mainly used for detecting charged particles generated by alpha radiation and has good energy resolution.

The high purity semiconductor detector is also generally referred to as a pure Ge detector and is a high-purity Ge crystal with very low impurity concentration and defects. The high purity semiconductor detector has very high electric resistance at low temperature and may also apply even a high bias voltage. The high purity semiconductor detector differs from Ge (Li) in that it may be stored at room temperature and may be used only by cooling it with liquid nitrogen only when it measures. Therefore, the high purity semiconductor detector is easy to maintain and energy resolution is practically not inferior to Ge (Li).

As for the scintillation detector, although the phenomenon that light is emitted when the charged particles collide with a certain substance has been known for a long time, the light emission by alpha-radiation of pieces of zinc sulfide (ZnS) or NaI is particularly strong and may be detected and counted by a magnifying glass in a dark room.

Such luminescence is called scintillation, and a substance showing this phenomenon is called a scintillator. In addition, a scintillator combined with a photomultiplier tube is called a scintillation detector, but, in particular, a scintillation detector with the method that a pulse output is used for the count is called a scintillation counter.

On the other hand, a detector taking the way of reading the output to DC is mainly used for dose measurement and is called a scintillation dosimeter because a scintillator is used therefor. The scintillator may be either solid, liquid, or gas, and when liquid is used, it is called a liquid scintillation counting device.

The solid state junction counter is a solid reverse bias p-n junction semiconductor, is a counter for collecting ion charge from alpha particles passing through a depletion layer, and may be manufactured in a small and mobile type.

The wireless communication unit 320 performs a function of wirelessly transmitting the radon concentration data for the corresponding specific indoor space measured by the indoor radon measurement sensor unit 310.

The indoor radon measurement controller 330 controls the overall operation of the indoor radon measurement module 300, may perform various functions for the indoor radon measurement module 300, and may execute or perform a set of various software programs and/or instructions stored in the storage unit 360 for processing the data. That is, the indoor radon measurement controller 330 may process various signals on the basis of the information stored in the storage unit 360.

In addition, the indoor radon measurement controller 330 may transmit and receive various signals to and from the wireless communication unit 320. That is, the indoor radon measurement controller 330 may perform various calculations on the basis of various signals transmitted and received to and from the wireless communication unit 320.

That is, the indoor radon measurement controller 330 controls the operation of the indoor radon measurement sensor unit 310, the wireless communication unit 320, the display unit 350, the storage unit 360, and the like. In particular, the indoor environment measurement controller 330 receives the radon concentration data for the corresponding specific indoor space measured from the indoor radon measurement sensor unit in real time, thereby controlling operations of the wireless communication unit 320 in order for the received radon concentration data to be wirelessly transmitted to the indoor radon prediction management server 500.

In addition, the indoor radon measurement controller 330 may perform a function of calculating the amount of the fine dust for the corresponding specific indoor space, when the indoor radon measurement sensor unit 310 measures radon, through the following equation 1 depending on the presence or absence of a filter (not shown) for separating radon progeny.

Total amount of radon=amount of pure radon+amount of radon progeny being attached to fine dust. (Equation 1)

Herein, the amount of pure radon is an amount of radon that is obtained by removing the amount of the radon progeny using a filter for separating the radon progeny when radon is measured, wherein the radon progeny is a substance produced when radon decays and is measured in a state of being attached to the fine dust.

That is, the pulsed ionization chamber radon measurement detector measures the alpha ray emitted from the radon gas in the indoor space. When radon is measured, the total amount of radon and the amount of pure radon are distinguished. The total amount of radon is obtained by calculating both the alpha ray emitted from the radon gas and the alpha ray emitted from the radon progeny produced after the radon gas decays.

Fundamentally, because the pulsed ionization chamber radon measurement detector measures all the alpha rays, when radon is measured, the amount of pure radon is measured by removing radon progeny, which is a substance produced when radon decays and is measured in a state of being attached to the fine dust, by using a HEPA filter to separate radon progeny.

With such characteristics of radon being used, the amount of the radon progeny may be identified by comparing the values measured by the radon measurement detector having a variable-type filter or a fixed-type filter and by the radon measurement detector without a filter.

The amount of fine dust in the indoor space may be measured through the above equation 1. Meanwhile, a conventional fine dust measurement method distinguishes the total amount of dust and the amount of fine dust by type by dividing the amount and size of dust by the light scattering method. However, because durability and maintenance of the optical sensor are difficult to secure, there is a practical difficulty to use it as a long term monitoring sensor.

When indoor dust prediction is made using the radon measurement sensor to solve this problem, it is difficult to distinguish kinds of dust but it may be used as a stable and durable fine dust measurement detector. In addition, it is very effective because it may accomplish radon measurement and fine dust prediction simultaneously with the radon measurement sensor.

The power supply unit 340 performs a function of supplying power necessary for the respective units, that is, the indoor radon measurement sensor unit 310, the wireless communication unit 320, the indoor environment measurement controller 330, the display unit 350, the storage unit 360, and the like. Therefore, the power supply unit 340 is preferable to be implemented to convert power source (e.g., AC 220V) to a DC and/or AC power source for continuous power supply but is not limited thereto. For example, the power supply unit 340 may be implemented with a solar power supply or a conventional portable battery.

For a manager to be able to performing monitoring, the display unit 350 performs a function of displaying the radon concentration data for the corresponding specific indoor space, wherein the environmental information data is measured from the indoor environment measurement sensor unit 310 according to the control of the indoor environment measurement controller 330.

The storage unit 360 may include a program memory and a data memory. The program memory stores programs for controlling the general operation of the indoor radon measurement module 300. At this time, the program memory may store a program for measuring radon concentration data for the corresponding specific indoor space through the indoor radon measurement module 300.

In addition, the program memory may store a program for driving the indoor radon measurement sensor unit 310, the wireless communication unit 320, the display unit 350, the storage unit 360, and the like under the control of the indoor radon measurement controller 330. The data memory stores data generated during the execution of the programs in the indoor radon measurement module 300. In the data memory, for example, module information, channel information, frequency information, network information, or the like may be stored. In addition, in the data memory of the storage unit 360, unique module identification information of the indoor radon measurement module 300 may be stored.

In addition, the storage unit 360 may store the radon concentration data for the corresponding specific indoor space, wherein the radon concentration data is measured from the indoor radon measurement sensor unit 310 according to the control of the indoor radon measurement controller 330.

That is, the storage unit 360 may store and maintain at least one program code executed through the indoor radon measurement controller 330 and at least one type of data set that the program code uses.

The KMA weather station management server 400 performs a function of constructing the database (DB) of the big data information for the surrounding weather conditions of a specific indoor space, thereby storing and managing the DB.

In addition, the big data information for the surrounding weather conditions of the specific indoor space stored and managed in the KMA weather management server 400 includes preferably at least one of information of temperature, humidity, atmospheric pressure, fine dust, rainfall, and snowfall.

The indoor radon prediction management server 500 receives the radon concentration data for the corresponding specific indoor space measured for a certain period of time from the indoor radon measurement module 300 and performs a function of generating an annual standard graph of the radon average concentration by time for the corresponding specific indoor space on the basis of the received radon concentration data.

In addition, the indoor radon prediction management server 500 reflects the big data information about the surrounding weather conditions for the corresponding specific indoor space managed by the KMA weather station management server 400 and the environmental information data measured from the soil environment measurement module 100 and the indoor environment measurement module 200, respectively, into the produced annual standard graph of the radon average concentration by time. In addition, the indoor radon prediction management server 500 performs a function of calculating the estimated radon measurement value by applying a correction index for each preset environmental element.

In addition, the indoor radon prediction management server 500 generates an hourly, daily, monthly, and/or yearly radon concentration prediction graph for the corresponding specific indoor space on the basis of the calculated estimated radon measurement value and constructs a DB for the radon concentration prediction graph, thereby performing a function of storing and managing the DB.

In addition, the indoor radon prediction management server 500 may perform a function of providing a management service so that the ventilation facility (not shown) is able to operate corresponding to the radon deviation for the corresponding indoor space according to the hourly, daily, monthly, and/or yearly produced radon concentration prediction graph for the corresponding specific indoor space.

In addition, the indoor radon prediction management server 500 may calculate the estimated radon measurement value by following equation 2.

Estimated radon measurement value=standard radon concentration measurement value×correction environment index. (Equation 2)

Herein, the standard radon concentration measurement value is a standardized value on a 24-hour basis for the corrected radon concentration measurement value over 48 hours, the corrected radon concentration measurement value is calculated as “radon concentration measurement value×environment index”, the radon concentration measurement value is calculated as “number of alpha rays×radon concentration conversion index”, the environment index is calculated as “temperature deviation index+humidity deviation index+fine dust deviation index+atmospheric pressure deviation index+rainfall index+snowfall index”, the temperature deviation index is calculated as “(indoor temperature−outdoor temperature)×temperature deviation weight”, the humidity deviation index is calculated as “outdoor humidity×humidity weight+(indoor humidity−outdoor humidity)×humidity deviation weight”, the fine dust deviation index is calculated as “indoor fine dust×fine dust weight+(outdoor fine dust−indoor fine dust)×fine dust deviation weight (when indoor fine dust<outdoor fine dust)”, the atmospheric pressure deviation index is calculated as (outdoor atmospheric pressure−indoor atmospheric pressure)×atmospheric pressure deviation weight”, the rainfall index is calculated as “rainfall×radon influence index×rainfall weight”, and the snowfall index is calculated as “snowfall×radon influence index×snowfall weight”, and the correction environment index is calculated as (1−current environment index/standard environment index)×environment weight”.

At this time, in the calculation of the fine dust deviation index, it is preferable that the fine dust deviation index is calculated as “indoor fine dust×fine dust weight” when the indoor fine dust>outdoor fine dust.

In addition, the indoor radon prediction management server 500 compares and analyzes the calculated estimated radon measurement value and the actual radon measurement value measured from the indoor radon measurement sensor unit 310 provided in the indoor radon measurement module 300 with each other, and, when a difference between the two values is greater than the preset reference deviation value, may perform a function of providing a management service to notify a regular calibration diagnosis time of the indoor radon measurement sensor unit 310 provided in the indoor radon measurement module 300 to the preset administrator terminal 20 through the communication network 10.

At this time, the communication network 10 is a communication network of a high speed backbone network of a large communication network capable of a large capacity, long distance voice and data service. Here, the communication network 10 may be a next-generation wireless communication network including a WiFi, a WiGig, and a Wireless Broadband Internet (Wibro), World Interoperability for Microwave Access (Wimx), and the like.

The Internet refers to a worldwide open computer network structure that provides a TCP/IP protocol and various services being existed on hierarchy thereabove, wherein the various services are such as a Hyper Text Transfer Protocol (HTTP), a Telnet, a File Transfer Protocol (FTP), a Domain Name System (DNS), a Simple Mail Transfer Protocol (SMTP), a Simple Network Management Protocol (SNMP), a Network File Service (NFS), a Network Information Service (NIS), and the like, and provides an environment that the administrator terminal 20 is able to be connected to the indoor radon prediction management server 500. Meanwhile, the Internet may be a wired or wireless Internet, or, besides above, may be a core network integrated with a wired public network, a wireless mobile communication network, or a portable Internet.

When the communication network 10 is a mobile communication network, it may be a synchronous mobile communication network or an asynchronous mobile communication network. As an example of the asynchronous mobile communication network, a Wideband Code Division Multiple Access (WCDMA) communication network may be exemplified. In this case, although not shown in the drawing, the mobile communication network may include, for example, a radio network controller (RNC) or the like. Meanwhile, although the WCDMA network is described as an example, the asynchronous mobile communication network may be a next-generation communication network such as a cellular-based 3G network, an LTE network, a 4G network, a 5G network, or other IP based IP network. The communication network 10 performs a function of mutually transferring signals and data between the administrator terminal 20 and the indoor radon prediction management server 500.

Meanwhile, the administrator terminal 20 applied to the embodiment of the present invention is preferable to be at least one mobile terminal device among a smartphone, a smart pad, and a smart note, which communicates through a wireless Internet or a portable Internet. Besides, the administrator terminal 20 may comprehensively mean all wired/wireless home appliances/communication devices having a user interface for accessing the indoor radon prediction management server 500 such as a personal computer, a notebook PC, a palm PC, a mobile play-station, a Digital Multimedia Broadcasting (DMB) phone with a communication function, a tablet PC, an iPad, and the like.

As illustrated in FIG. 5, the administrator terminal 20 may include a wireless communication module 21, an audio/video (A/V) input module 22, a user input module 23, a sensing module 24, an output module 25, a storage module 26, an interface module 27, a terminal control module 28, a power module 29, and the like. On the other hand, because the elements illustrated in FIG. 5 are not essential, the administrator terminal 20 may have more or fewer elements than the administrator terminal 20 illustrated in FIG. 5.

Hereinafter, the elements of the administrator terminal 20 will be described in detail.

The wireless communication module 21 may include one or more modules enabling wireless communication between the administrator terminal 20 and the indoor radon prediction management server 500. For example, the wireless communication module 21 may include a broadcast receiving module 21a, a mobile communication module 21b, a wireless Internet module 21c, a short-range communication module 21d, and a location information module 21e, and the like.

The broadcast receiving module 21a receives a broadcast signal (e.g., a TV broadcast signal, a radio broadcast signal, a data broadcast signal, etc.) and/or the broadcast-related information from an external broadcast management server through various broadcast channels (e.g., a satellite channel and a terrestrial channel, etc.).

The mobile communication module 21b transmits and receives a radio signal to and from at least one of a base station, an external terminal, and a server on a mobile communication network. The radio signal may include various types of data according to a voice call signal, a video communication call signal, or a text/multimedia message transmission/reception.

The wireless Internet module 21c is a module for wireless Internet access, and may be embedded in the administrator terminal 20 or mounted externally. As the wireless Internet technology, for example, WLAN (Wi-Fi), Wibro, Wimax, HSDPA, LTE and the like may be used.

The short-range communication module 21d is a module for short-range communication, and Bluetooth communication, ZigBee communication, UWB (Ultra Wideband) communication, RFID (Radio Frequency Identification) communication, and the like may be used.

The position information module 21e is a module for confirming or obtaining the position of the administrator terminal 20, and may acquire current position information of the administrator terminal 20 using a global position system (GPS) or the like.

Meanwhile, in accordance with the control of the terminal control module 28, the administrator terminal 20 may perform data transmission/reception with the mobile station 500 using the specific application program stored in the storage module 26 through the wireless communication module 21 and/or the wired communication module (not shown).

An audio/video (A/V) input module 22 is a module for inputting an audio signal or a video signal, and may fundamentally include a camera section 22a and a microphone section 22b. The camera section 22a processes an image frame such as a still image or a moving image obtained by the image sensor in the video communication mode or the photographing mode. The microphone section 22b receives an external sound signal by a microphone in a communication mode, a recording mode, a voice recognition mode, or the like, and processes it as electrical voice data.

The user input module 23 is a module for generating input data for controlling the operation of the administrator terminal 20 and, in particular, performs a function of inputting a selection signal for one of the network monitoring information displayed through the display portion 25a of the output module 25. For example, the user input module 23 is a touch panel (static pressure/static electricity) type input by a user's touch or may be input using a separate input device (e.g., a keypad dome switch, a jog wheel, a jog switch, etc.).

The sensing module 24 detects the current state of the administrator terminal 20 such as the open/close state of the administrator terminal 20, the position of the administrator terminal 20, the presence or absence of user contact, the user's touching operation on a specific part, the orientation of the administrator terminal 20, the acceleration/deceleration of the administrator terminal 20, and the like, thereby generating a sensing signal for controlling the operation of the administrator terminal 20. The sensing signal is transmitted to the terminal control module 28, and the terminal control module 28 may be a basis for performing a specific function.

The output module 25 is a module for generating an output related to visual, auditory or tactile sense and may fundamentally include a display portion 25a, a sound output portion 25b, an alarm portion 25c, a haptic portion 25d.

The display portion 25a is for displaying and outputting information processed by the administrator terminal 20. For example, the display portion 25a displays a User Interface (UI) or Graphical User Interface (GUI) when the administrator terminal 20 is in the call mode and displays the photographed and/or received image or the UI and the GUI when the administrator terminal 20 is in the video communication mode or the photographing mode.

The sound output portion 25b may also output audio data, which is received from the wireless communication module 21 or is stored in the storage module 26 when the sound output portion 25b is in, for example, a call signal reception mode, a communication mode or a recording mode, a voice recognition mode, a broadcast reception mode, and the like.

The alarm portion 25c may output a signal for notifying an event occurred in the administrator terminal 20. Examples of the event generated in the administrator terminal 20 may be call signal reception, message reception, key signal input, touch input, and the like.

The haptic portion 25d generates various tactile effects that the user may feel. A typical example of the haptic effect generated by the haptic portion 25d is vibration. The intensity and pattern of the vibration generated by the haptic portion 25d may be controlled.

The storage module 26 may store a program for the operation of the terminal control module 28 as well as temporarily store a number of data (e.g., a phone book, a message, a still image, a moving image, etc.) being input/output.

In addition, the storage module 26 may store data related to vibrations and sounds of various patterns, which are output when a touch is input on the touch screen and may store the notification related application program of the indoor radon prediction management server 500.

In addition, the storage module 26 may store the source data for forming the notification related information of the indoor radon prediction management server 500 and, therefore, may be configured in a form that the notification related data of the indoor radon prediction management server is composed of video and sound. In addition, the storage module 26 may store together with the process and result of generating the notification related data of the indoor radon prediction management server 500.

Such a storage module 26 may include at least one type of memory medium among a memory (for example, SD or XD memory), a RAM, an SRAM, a ROM, an EEPROM, a PROM, a magnetic memory, a magnetic disk, an optical disk, and the like, wherein the memory is a flash memory type, a hard disk type, a multimedia card micro type, and a card type.

The interface module 27 plays a role as a path for communication with all the external devices connected to the administrator terminal 20. The interface module 27 receives data or power from an external device and transfers the data or power to the respective elements in the administrator terminal 20 or allows data in the administrator terminal 20 to be transmitted to the external device.

The terminal control module 28 generally controls the overall operation of the administrator terminal 20 and performs related control and processing for, for example, voice calls, data communication, video call, and execution of various applications.

That is, the terminal control module 28 performs a function of controlling the notification related application program of the indoor radon prediction management server 500, which is stored in the storage module 26, to be executed, requesting generation of the notification related data of the indoor radon prediction management server 500 through execution of the notification related application program of the indoor radon prediction management server 500, and controlling to be provided with the notification related data of the indoor radon prediction management server 500 in response to the request.

In addition, the terminal control module 28 performs a function of controlling auxiliary elements including at least one of video, audio, and sound, which the user desires and is produced in the process of generating the notification related data of the indoor radon prediction management server 500 through execution of the notification related application program of the indoor radon prediction management server 500, to be output through at least one of the display portion 25a and other output devices (e.g., the sound output portion 25b, the alarm portion 25c, the haptic portion 25d, etc.)

In addition, the terminal control module 28 may regularly monitor the charging current and the charging voltage of the battery unit 29a and temporarily store the monitoring value in the storage module 26. At this time, the storage module 26 preferably stores not only the battery charging status information such as the monitored charging current and the charging voltage, but also battery specification information (product code, rated value, etc.).

The power supply module 29 receives external power and internal power under the control of the terminal control module 28 and supplies power necessary for operation of the respective elements. The power module 29 supplies the power of the built-in battery unit 29a to the respective elements to operate, and the battery may be charged using a charging terminal (not shown).

The various embodiments described herein may be embodied in a recording medium readable by a computer or a similar device using, for example, software, hardware, or a combination thereof.

According to a hardware implementation, the embodiments described herein may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, the electrical units for performing functions, and the like. In some cases, such embodiments may be implemented by the terminal control module 28.

According to a software implementation, embodiments such as procedures or functions may be implemented with separate software modules allowing at least one function or operation to be performed. The software code may be implemented by a software application written in a suitable programming language. The software code may also be stored in the storage module 26 and executed by the terminal control module 28.

On the other hand, when the administrator terminal 20 is a smartphone, unlike a general mobile phone (so-called a feature phone), the smartphone is a phone based on the open operating system in which the user is capable of downloading desired various application programs, thereby using or deleting the application programs freely. In other words, the smartphone is to be preferably understood to include: all mobile phones having not only a commonly used voice/video call function, Internet data communication, and the like but also a mobile office function; or communication devices not having a voice call function but including all Internet-enabled Internet phones or tablet PCs.

Such smartphone may be implemented as a smartphone equipped with various open operating systems. Examples of the open operating system include Symbian of Nokia, BlackBerry of RIM's, iPhone of Apple, Windows Mobile of Microsoft, Android of Google, and Ocean of Samsung.

As such, because the smartphone uses an open operating system, a user may arbitrarily install and manage various application programs, unlike a mobile phone having a closed operating system.

That is, the smartphone described above fundamentally is provided with a controller, a memory unit, a screen output unit, a key input unit, a sound output unit, a sound input unit, a camera unit, a wireless network communication module, a short-range wireless communication module, and a battery for supplying power.

Such a controller is a generic term for a functional configuration for controlling the operation of the smartphone, includes at least one processor and an execution memory, and is connected to each functional unit provided in the smartphone through a bus.

The controller calculates by loading at least one program code provided in the smartphone into the execution memory through the processor and controls the operation of the smartphone by transmitting the calculating result to the at least one functional unit through the bus.

The memory unit is a general term of a nonvolatile memory provided in a smartphone, and stores and maintains at least one program code executed through the controller and at least one data set used by the program code. The memory unit fundamentally stores a system program code, which corresponds to an operating system of a smartphone, and a system data set, a communication program code, which processes a wireless communication connection of the smartphone, and a communication data set, and at least one application program code and an application data set. In addition, the program code and the data set for implementing the present invention are also stored in the memory unit.

The screen output unit includes a screen output device (e.g., an Liquid Crystal Display (LCD) device) and an output module for driving the screen output device. In addition, the screen output unit is connected to the controller through a bus, and outputs a calculation result corresponding to a screen output among various calculation results of the controller to the screen output device.

The key input unit comprises a key input device (or a touch screen device being interlocked with the screen output unit) having at least one key button and an input module driving therefor. In addition, the key input unit is connected to the controller through a bus, thereby inputting commands commanding various calculations of the controller or inputting data necessary for the calculation of the controller.

The sound output unit is composed of a speaker outputting a sound signal and a sound module driving therefor. In addition, the sound output unit is connected to the controller through a bus and outputs calculation results corresponding to the sound output among the various calculation results of the controller. The sound module decodes sound data to be outputted through the speaker, thereby converting the sound data into a sound signal.

The sound input unit is composed of a microphone receiving a sound signal and a sound module driving therefor and transmits the sound data input through the microphone to the controller. The sound module encodes the sound signal via encoding the sound signal input through the microphone.

The camera unit is composed of an optical unit and a Charge Coupled Device (CCD) and a camera module driving therefor and obtains bitmap data input to the CCD through the optical unit. The bitmap data may include both still image data and moving image data

The wireless network communication module is a collective term for communication construction connecting wireless communication and includes at least one antenna, an RF module, a baseband module, and a signal processing module for transmitting and receiving a radio frequency signal of a specific frequency band. In addition, the wireless network is connected to the controller with a bus and transmits the calculation result corresponding to the wireless communication among the various calculation results of the controller through the wireless communication or receives data through the wireless communication, thereby transmitting the data to the controller and, at the same time, maintains the connection, registration, communication, and handoff procedures of the wireless communication.

In addition, the wireless network communication module includes a mobile communication structure performing at least one of connection, location registration, call processing, call connection, data communication, and handoff to a mobile communication network according to the CDMA/WCDMA standard. Meanwhile, according to the intention of the person skilled in the art, the wireless network communication module may further include a portable Internet communication structure performing at least one of connection, location registration, data communication and handoff to the portable Internet according to the IEEE 802.16 standard. It is evident that the present invention is not limited by the wireless communication configuration provided by the wireless network communication module.

The short-range wireless communication module is composed of a short-range wireless communication module that connects a communication session using a radio frequency signal as a communication medium within a predetermined distance. The short-range wireless communication module preferably includes RFID communication, Bluetooth communication, Wi-Fi communication, and airborne radio communication satisfying ISO 180000 series standard. In addition, the short-range wireless communication module may be integrated with the wireless network communication module.

The smartphone configured as such means a terminal capable of wireless communication, and any device including a terminal capable of transmitting and receiving data through a network including the Internet as well as a smart phone may be applicable. That is, the smart phone may include at least one of a notebook PC having a short message transmission function and a network connection function, a tablet PC as well as a portable terminal capable of being carried and moved.

Hereinafter, an indoor radon prediction method for radon reduction according to an embodiment of the present invention will be described in detail.

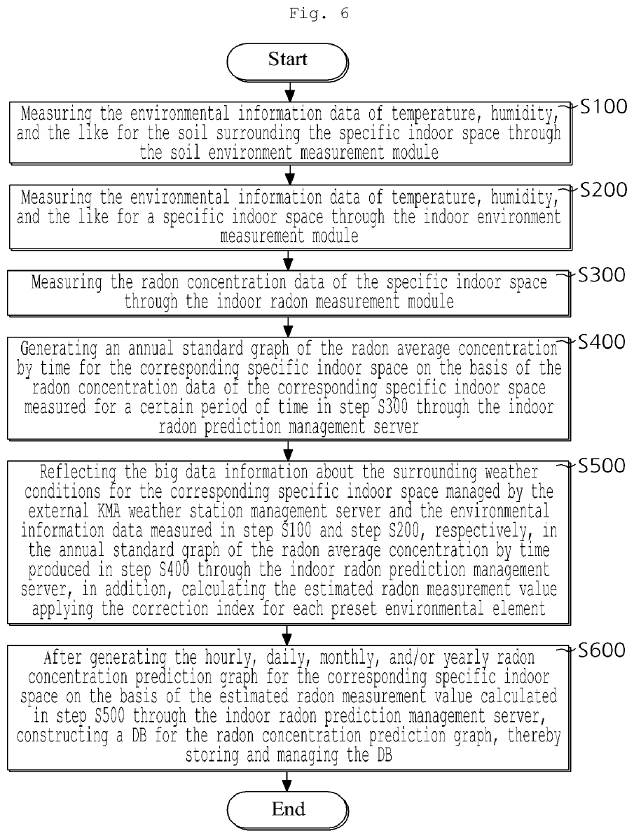

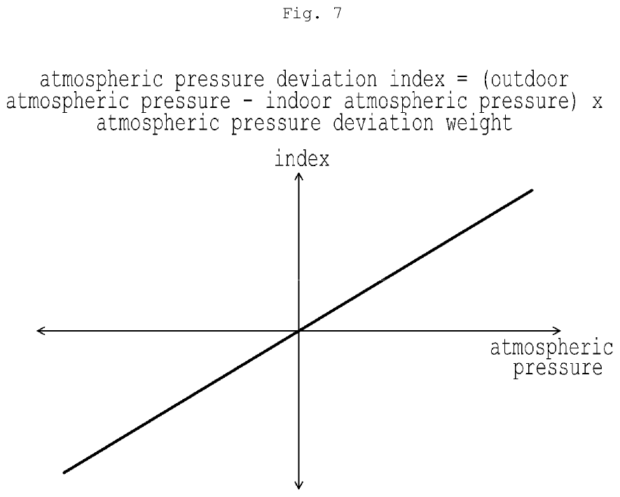

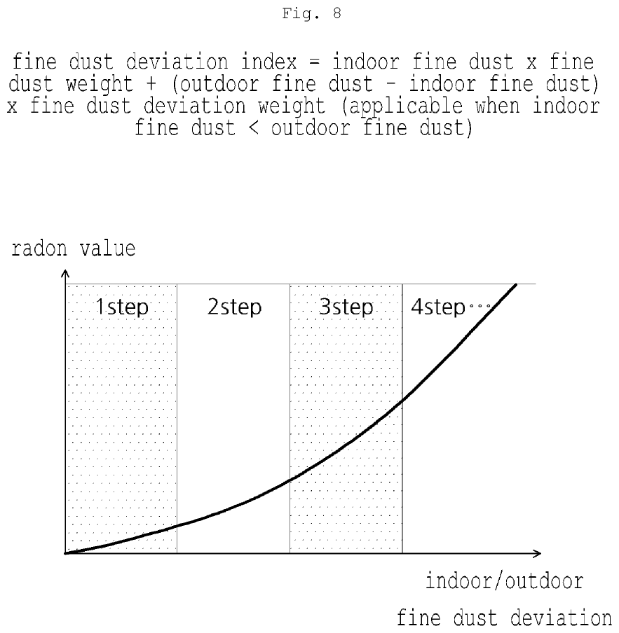

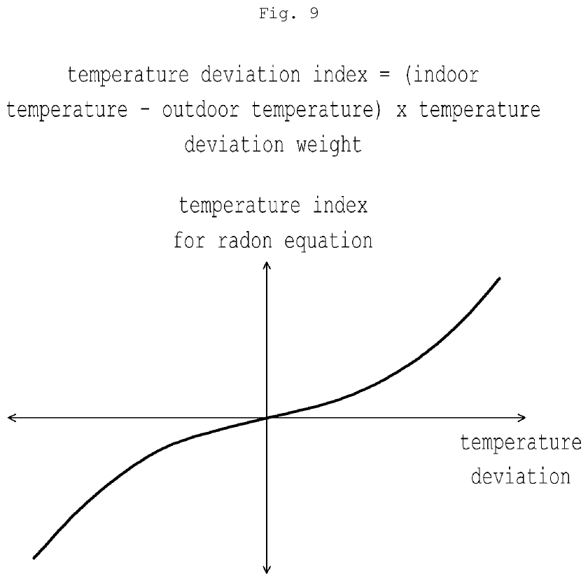

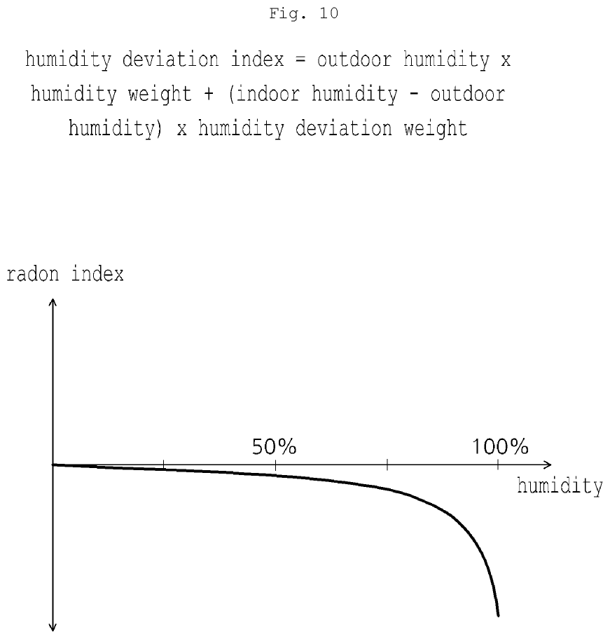

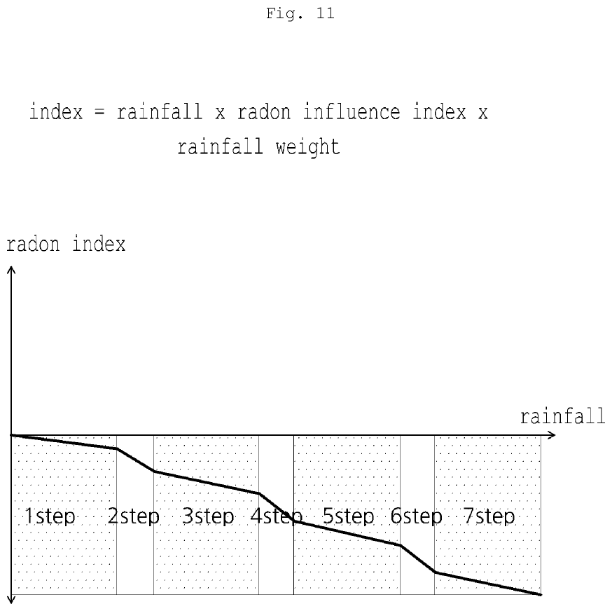

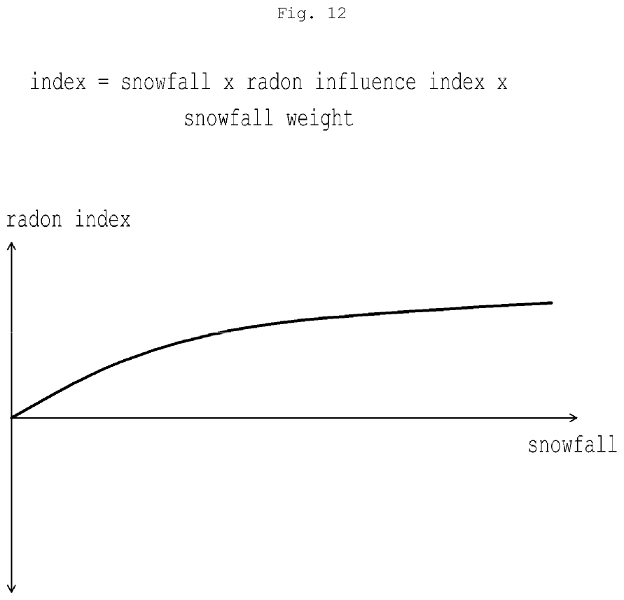

FIG. 6 is an overall flowchart for illustrating an indoor radon prediction system for radon reduction according to an embodiment of the present invention. FIGS. 7 to 12 are views in graphs each illustrating environmental indices influencing indoor radon prediction for radon reduction according to an embodiment of the present invention.

With reference to FIGS. 1 to 12, an indoor radon prediction method for radon reduction according to an embodiment of the present invention, first, in S100, measures the environmental information data of temperature, humidity, and the like for the soil surrounding the specific indoor space through the soil environment measurement module 100.

Thereafter, in S200, the method measures environmental information data of temperature, humidity, and the like for a specific indoor space through the indoor environment measurement module 200.

Then, in S300, the method measures the radon concentration data of the specific indoor space through the indoor radon measurement module 300.

Next, in S400, the method generates an annual standard graph of the radon average concentration by time for the corresponding specific indoor space on the basis of the radon concentration data of the corresponding specific indoor space measured for a certain period of time in step S300 through the indoor radon prediction management server 500.

Thereafter, in S500, the method reflects the big data information about the surrounding weather conditions for the corresponding specific indoor space managed by the external KMA weather station management server 400 and the environmental information data measured in step S100 and step S200, respectively, in the annual standard graph of the radon average concentration by time produced in step S400 through the indoor radon prediction management server 500, in addition, calculates the estimated radon measurement value applying the correction index for each preset environmental element.

At this time, in step S500, the indoor radon prediction management server 500 may calculate the estimated radon measurement value according to following equation 3.

Estimated radon measurement value=standard radon concentration measurement value×correction environment index. (Equation 3)

Herein, the standard radon concentration measurement value is a standardized value on a 24-hour basis for the corrected radon concentration measurement value over 48 hours, the corrected radon concentration measurement value is calculated as “radon concentration measurement value×environment index”, the radon concentration measurement value is calculated as “number of alpha rays×radon concentration conversion index”, the environment index is calculated as “temperature deviation index+humidity deviation index+fine dust deviation index+atmospheric pressure deviation index+rainfall index+snowfall index”, the temperature deviation index is calculated as “(indoor temperature−outdoor temperature)×temperature deviation weight”, the humidity deviation index is calculated as “outdoor humidity×humidity weight+(indoor humidity−outdoor humidity)×humidity deviation weight”, the fine dust deviation index is calculated as “indoor fine dust×fine dust weight+(outdoor fine dust−indoor fine dust)×fine dust deviation weight (when indoor fine dust<outdoor fine dust)”, the atmospheric pressure deviation index is calculated as (outdoor atmospheric pressure−indoor atmospheric pressure)×atmospheric pressure deviation weight”, the rainfall index is calculated as “rainfall×radon influence index×rainfall weight”, and the snowfall index is calculated as “snowfall×radon influence index×snowfall weight”, and the correction environment index is calculated as (1−current environment index/standard environment index)×environment weight”.

At this time, in the calculation of the fine dust deviation index, it is preferable that fine dust deviation index is calculated as “indoor fine dust×fine dust weight” when the indoor fine dust>outdoor fine dust.

Meanwhile, in step S500, the big data information about the ambient weather conditions for the specific indoor space stored and managed in the external KMA weather station management server 400 preferably includes at least one of temperature, humidity, air pressure, fine dust, rainfall, and snowfall.

Then, in S600, after generating the hourly, daily, monthly, and/or yearly radon concentration prediction graph for the corresponding specific indoor space on the basis of the estimated radon measurement value calculated in step S500 through the indoor radon prediction management server 500, the method constructs a DB for the radon concentration prediction graph, thereby being stored and managed.

In addition, although not illustrated in the drawing, after step S500, through the indoor radon prediction management server 500, the estimated radon measurement value calculated in step S500 and the actual radon measurement value measured from the indoor radon measurement sensor unit 310 provided in the indoor radon measurement module 300 are compared with each other and analyzed. When a difference between the two values is greater than the preset reference deviation value, the method may further include a step of providing a management service to notify a regular calibration diagnosis time of the indoor radon measurement sensor unit 310 provided in the indoor radon measurement module 300 to the preset administrator terminal 20.

Further, after step S600, the method may further include a step of providing a management service so that the ventilation facility is able to operate corresponding to the radon deviation for the corresponding indoor space according to the hourly, daily, monthly, and/or yearly radon concentration prediction graph for the corresponding specific indoor space produced in step S600 through the indoor radon prediction management server 500.

Although the present invention has been described with respect to a preferred embodiment of the indoor radon prediction system and method for radon reduction according to the present invention, the present invention is not limited thereto. In addition, it is possible to carry out various modifications within the scope of the claims, detailed description of the present invention and the accompanying drawings, wherein such modifications also belong to the present invention.

### INDUSTRIAL APPLICABILITY

The present invention may be widely used in radon prediction systems.

1. An indoor radon prediction system for radon reduction, the system comprising:
a soil environment measurement module installed in soil surrounding a specific indoor space, and measuring environmental information data of temperature and humidity for the soil surrounding the corresponding specific indoor space;
an indoor environment measurement module installed in the specific indoor space and measuring environmental information data of temperature and humidity for the corresponding specific indoor space;
an indoor radon measurement module installed in the specific indoor space and measuring radon concentration data of the corresponding specific indoor space;
a Korea Meteorological Administration (KMA) weather station management server constructing a database (DB) of big data information for surrounding weather conditions of the specific indoor space, thereby storing and managing the DB; and
an indoor radon prediction management server receiving the radon concentration data for the corresponding specific indoor space measured for a certain period of time from the indoor radon measurement module, generating an annual standard graph of the radon average concentration by time for the corresponding specific indoor space on the basis of the received radon concentration data, reflecting the big data information about the surrounding weather conditions for the corresponding specific indoor space managed by the KMA weather station management server and the environmental information data measured from the soil environment measurement module and the indoor environment measurement module, respectively, in the produced annual standard graph of the radon average concentration by time, in addition, calculating the estimated radon measurement value applying a correction index for each preset environmental element, generating an hourly, daily, monthly, and yearly radon concentration prediction graph for the corresponding specific indoor space on the basis of the calculated estimated radon measurement value, and constructing a DB for the radon concentration prediction graph, thereby storing and managing the DB.

2. The system of claim 1, wherein the soil environment measurement module includes:
a soil environment measurement module installed in the soil surrounding the specific indoor space and measuring environmental information data of temperature and humidity for the soil surrounding the corresponding specific indoor space;
a wireless communication unit wirelessly transmitting environmental information data of temperature and humidity for the soil surrounding the corresponding specific indoor space measured from the soil environment measurement sensor unit; and
a soil environment measurement controller receiving the environmental information data of temperature and humidity for the soil surrounding the corresponding specific indoor space measured from the soil environment measurement sensor unit in real time, thereby controlling operations of the wireless communication unit in order for the received environmental information data to be wirelessly transmitted to the indoor radon prediction management server.

3. The system of claim 1, wherein the indoor environment measurement module includes:
an indoor environment measurement sensor unit installed in a specific indoor space and measuring environmental information data of temperature and humidity for the corresponding specific indoor space;
a wireless communication unit wirelessly transmitting environmental information data of temperature and humidity for the specific indoor space measured from the indoor environment measurement sensor unit; and
an indoor environment measurement controller receiving the environmental information data of temperature and humidity for the corresponding specific indoor space measured from the indoor environment measurement sensor unit in real time, thereby controlling operations of the wireless communication unit in order for the received environmental information data to be wirelessly transmitted to the indoor radon prediction management server.

4. The system of claim 1, wherein the indoor radon measurement module includes:
an indoor radon measurement sensor unit installed in a specific indoor space and measuring radon concentration data of the corresponding specific indoor space;
a wireless communication unit wirelessly transmitting the radon concentration data for the corresponding specific indoor space measured from the indoor radon measurement sensor unit; and
an indoor radon measurement controller receiving the radon concentration data for the corresponding specific indoor space measured from the indoor radon measurement sensor unit in real time, thereby controlling operations of the wireless communication unit in order for the received radon concentration data to be wirelessly transmitted to the indoor radon prediction management server.

5. The system of claim 4, wherein the indoor radon measurement sensor unit is composed of a pulsed ionization chamber radon measurement sensor.

6. The system of claim 4, wherein, when the indoor radon measurement sensor unit measures radon, the indoor radon measurement controller calculates the amount of fine dust for the corresponding specific indoor space through following equation 1 depending on presence or absence of a filter for separating radon progeny,
total amount of radon=amount of pure radon+amount of radon progeny being attached to fine dust, (Equation 1)
wherein, the amount of pure radon is an amount of radon that is obtained by removing the amount of the radon progeny using a filter for separating the radon progeny when radon is measured, wherein the radon progeny is a substance produced when radon decays and is measured in a state of being attached to the fine dust.

7. The system of claim 1, wherein the big data information for the surrounding weather conditions of the specific indoor space stored and managed in the KMA weather management server includes at least one of information of temperature, humidity, atmospheric pressure, fine dust, rainfall, and snowfall.

8. The system of claim 1, wherein the indoor radon prediction management server compares and analyzes the calculated estimated radon measurement value and the actual radon measurement value measured from the indoor radon measurement sensor unit provided in the indoor radon measurement module with each other, and, when a difference between the two values is greater than the preset reference deviation value, provides a management service to notify a regular calibration diagnosis time of the indoor radon measurement sensor unit provided in the indoor radon measurement module to the preset administrator terminal through the communication network.

9. The system of claim 1, wherein the indoor radon prediction management server provides a management service so that the ventilation facility is able to operate corresponding to the radon deviation for the corresponding indoor space according to the produced hourly, daily, monthly, and yearly radon concentration prediction graph for the corresponding specific indoor space.

10. The system of claim 1, wherein the indoor radon prediction management server calculates the estimated radon measurement value by following equation 2,
estimated radon measurement value=standard radon concentration measurement value×correction environment index, (Equation 2)
wherein, the standard radon concentration measurement value is a standardized value on a 24-hour basis for the corrected radon concentration measurement value over 48 hours, the corrected radon concentration measurement value is calculated as “radon concentration measurement value×environment index”, and the radon concentration measurement value is calculated as “radon concentration measurement value×environment index”, the radon concentration measurement value is calculated as “number of alpha rays×radon concentration conversion index”, the environment index is calculated as “temperature deviation index+humidity deviation index+fine dust deviation index+atmospheric pressure deviation index+rainfall index+snowfall index”, the temperature deviation index is calculated as “(indoor temperature−outdoor temperature)×temperature deviation weight”, the humidity deviation index is calculated as “outdoor humidity×humidity weight+(indoor humidity−outdoor humidity)×humidity deviation weight”, the fine dust deviation index is calculated as “indoor fine dust×fine dust weight+(outdoor fine dust−indoor fine dust)×fine dust deviation weight (when indoor fine dust<outdoor fine dust)”, the atmospheric pressure deviation index is calculated as (outdoor atmospheric pressure−indoor atmospheric pressure)×atmospheric pressure deviation weight”, the rainfall index is calculated as “rainfall×radon influence index×rainfall weight”, and the snowfall index is calculated as “snowfall×radon influence index×snowfall weight”, and the correction environment index is calculated as (1−current environment index/standard environment index)×environment weight”.

11. The system of claim 10, wherein, when the indoor fine dust>outdoor fine dust, the fine dust deviation index is calculated as “indoor fine dust×fine dust weight”.

12. An indoor radon prediction method for radon reduction as the method using a system comprising a soil environment measurement module, an indoor environment measurement module, an indoor radon measurement module, and an indoor radon prediction management server, the method comprising:
step (a) of measuring environmental information data of temperature and humidity for soil surrounding specific indoor space through the soil environment measurement module;
step (b) of measuring environmental information data of temperature and humidity for the specific indoor space through the indoor environment measurement module;
step (c) of measuring radon concentration data of the specific indoor space through the indoor radon measurement module;
step (d) of generating an annual standard graph of the radon average concentration by time for the corresponding specific indoor space on the basis of the radon concentration data of the corresponding specific indoor space measured for a certain period of time in step (c) through the indoor radon prediction management server;
step (e) of reflecting the big data information about the surrounding weather conditions for the corresponding specific indoor space managed by an external Korea Meteorological Administration (KMA) weather station management server and the environmental information data measured in step (a) and step (b), respectively, into the annual standard graph of the radon average concentration by time produced in step (d) through the indoor radon prediction management server, in addition, calculating estimated radon measurement value applying a correction index for each preset environmental element; and
step (f) of, after generating the hourly, daily, monthly, and yearly radon concentration prediction graph for the corresponding specific indoor space on the basis of the estimated radon measurement value calculated in step (e) through the indoor radon prediction management server, constructing a DB for the radon concentration prediction graph, thereby storing and managing the DB.

13. The method of claim 12, wherein, in step (e), the big data information for the surrounding weather conditions of the specific indoor space stored and managed in the external KMA weather management server includes at least one of information of temperature, humidity, atmospheric pressure, fine dust, rainfall, and snowfall.

14. The method of claim 12, wherein, after step (e), when the estimated radon measurement value calculated in step (e) and the actual radon measurement value measured from the indoor radon measurement sensor unit provided in the indoor radon measurement module are compared and analyzed through the indoor radon prediction management server, and, a difference between the two values is greater than the preset reference deviation value, the method further includes a step of providing a management service to notify a regular calibration diagnosis time of the indoor radon measurement sensor unit provided in the indoor radon measurement module to the preset administrator terminal through the communication network.

15. The method of claim 12, wherein, after step (f), the method further include a step of providing a management service so that the ventilation facility is able to operate corresponding to the radon deviation for the corresponding indoor space according to the hourly, daily, monthly, and yearly radon concentration prediction graph for the corresponding specific indoor space produced in step (f) through the indoor radon prediction management server.

16. The method of claim 12, wherein, in step (e), the indoor radon prediction management server calculates the estimated radon measurement value according to following equation 3,
estimated radon measurement value=standard radon concentration measurement value×correction environment index, (Equation 3)
wherein, the standard radon concentration measurement value is a standardized value on a 24-hour basis for the corrected radon concentration measurement value over 48 hours, the corrected radon concentration measurement value is calculated as “radon concentration measurement value×environment index”, the radon concentration measurement value is calculated as “number of alpha rays×radon concentration conversion index”, the environment index is calculated as “temperature deviation index+humidity deviation index+fine dust deviation index+atmospheric pressure deviation index+rainfall index+snowfall index”, the temperature deviation index is calculated as “(indoor temperature−outdoor temperature)×temperature deviation weight”, the humidity deviation index is calculated as “outdoor humidity×humidity weight+(indoor humidity−outdoor humidity)×humidity deviation weight”, the fine dust deviation index is calculated as “indoor fine dust×fine dust weight+(outdoor fine dust−indoor fine dust)×fine dust deviation weight (when indoor fine dust<outdoor fine dust)”, the atmospheric pressure deviation index is calculated as (outdoor atmospheric pressure−indoor atmospheric pressure)×atmospheric pressure deviation weight”, the rainfall index is calculated as “rainfall×radon influence index×rainfall weight”, and the snowfall index is calculated as “snowfall×radon influence index×snowfall weight”, and the correction environment index is calculated as (1−current environment index/standard environment index)×environment weight”.

17. The method of claim 16, wherein, when the indoor fine dust>the outdoor fine dust, the fine dust deviation index is calculated as “indoor fine dust×fine dust weight”.
